(19) United States
(12) Patent Application Publication
(10) Pub. No.: US 2025/0152207 A1
(43) Pub. Date: May 15, 2025

(54) LATERAL MASS FIXATION IMPLANT

(71) Applicant: PROVIDENCE MEDICAL TECHNOLOGY, INC., Pleasanton, CA (US)

(72) Inventors: Bruce M. McCormack, San Francisco, CA (US); Edward Liou, Pleasanton, CA (US); Shigeru Tanaka, Half Moon Bay, CA (US); Christopher U. Phan, Dublin, CA (US); Jeffrey D. Smith, Clayton, CA (US); Todd Sheppard Saunders, Walnut Creek, CA (US); Krzysztof Siemionow, Chicago, IL (US)

(21) Appl. No.: 19/024,356

(22) Filed: Jan. 16, 2025

Related U.S. Application Data
(63) Continuation of application No. 18/653,309, filed on May 2, 2024.
Application No. 18/653,309 is a continuation of application No. 16/997,971, filed on Aug. 20, 2020, now Pat. No. 12,004,781.
Application No. 16/997,971 is a continuation of application No. 14/723,243, filed on May 27, 2015, now abandoned.
(60) Provisional application No. 62/003,443, filed on May 27, 2014.

(51) Int. Cl. A61B 17/70; A61B 17/56

(52) U.S. Cl. CPC A61B 17/7064; A61B 17/7002; A61B 2017/564; A61B 17/7035

(57) ABSTRACT

The various embodiments described herein provide lateral mass and facet fixation implants, which may be inserted and applied via a posterior approach, using minimally invasive or less invasive techniques. The embodiments described below generally include an intrafacet implant (or “facet implant”) and a lateral mass fixation member attached to or attachable to the facet implant. The lateral mass fixation member can include one or more tabs extending from a middle portion and configured to secure the lateral mass fixation member to lateral masses of adjacent vertebrae. The tabs may be flexible, semi-rigid, or rigid, and may be collapsible to facilitate insertion of the device. Methods for delivering the lateral mass and facet fixation implants are also described.

### CROSS-REFERENCE TO RELATED APPLICATIONS

This application is a continuation of U.S. patent application Ser. No. 18/653,309, filed May 2, 2024, which is a continuation of U.S. patent application Ser. No. 16/997,971, filed Aug. 20, 2020, now U.S. Pat. No. 12,004,781, issued Jun. 11, 2024, which is a continuation of U.S. patent application Ser. No. 14/723,243, filed May 27, 2015, which claims priority to U.S. Provisional Patent Application No. 62/003,443, filed on May 27, 2014, all titled “LATERAL MASS FIXATION IMPLANT.” The full disclosures of the above-listed patent applications are hereby incorporated by reference in their entireties and for all purposes.

### BACKGROUND

Posterior cervical fusion with lateral mass fixation is the most rigid cervical instrumentation. It requires extensive dissection of muscles and ligaments off the posterior spine, so that the surgeon can have direct visualization to safely perform the procedure. This dissection causes acute and chronic soft tissue pain syndrome. Acutely, patients are typically hospitalized for three to four days for pain control that requires IV narcotics. This is compared to one-day hospitalization for anterior approaches that do not require any muscle or soft tissue dissection. Long-term patients with posterior approaches frequently have persistent pain due to the extensive nature of the dissection. Sometimes, after posterior-access cervical fusion surgery, soft tissues may not return to anatomic position and may be permanently deformed. Persistent pain after posterior surgical approaches is referred to as post-laminectomy syndrome. (FIG. 1 is a lateral view of the C5 and C6 cervical vertebrae, illustrating the anatomy.)

Therefore, since it is considered less traumatic to the patient compared to posterior approaches, anterior cervical spinal fusion surgery has generally been preferred over posterior fusion surgery. At the same time, posterior approaches to the cervical spine do have some advantages over anterior approaches.

Lateral mass or pedicle screw fixation provides more rigid fixation of the cervical spine than anterior plates, interbody devices and interspinous wiring. It is best for traumatic instability, but it has also been used for degenerative conditions. Despite being the best fixation, lateral mass fixation is often avoided, because of the morbidity of the soft tissue dissection, as noted above. (FIGS. 2A and 2B are posterior and lateral views, respectively, of a cervical spine with posterior fixation devices applied thereto.)

Starting a drill hole or inserting a screw into a lateral mass of a vertebra cannot currently be accomplished using a percutaneous approach. This is because soft tissue gets caught up in the drill, and the drill can skid off the bone and go out of control. Awls and firm pressure placed on bone with screws without direct visualization is dangerous in the posterior cervical spine, unless the surgeon has removed soft tissue and has direct visualization.

Therefore, it would be advantageous to have improved devices, systems and methods for performing cervical spinal fusion procedures via posterior access approaches. Ideally, these devices, systems and methods would allow for minimally invasive or less invasive access and fixation, as well as helping ensure proper placement of the fixation devices. At least some of these objectives will be met by the embodiments described herein.

### BRIEF SUMMARY

The various embodiments described herein provide lateral mass and facet fixation implants, which may be inserted and applied via a posterior approach, using minimally invasive or less invasive techniques. The embodiments described below generally include an intrafacet implant (or “facet implant”) and a lateral mass fixation member attached to or attachable to the facet implant. The lateral mass fixation member can include one or more tabs extending from a middle portion and configured to secure the lateral mass fixation member to lateral masses of adjacent vertebrae. The tabs may be flexible, semi-rigid, or rigid, and may be collapsible to facilitate insertion of the member.

In one aspect, a spinal implant system is disclosed. The spinal implant system includes a facet implant member for positioning in a facet joint and a lateral mass fixation member attached to or attachable to the facet implant member. The facet implant member and the lateral mass fixation member may be two separate devices or components that are attachable in situ or they may be a single device or of a single piece or monolithic construction. In some embodiments, the facet implant member includes a peg and the lateral mass fixation member includes a hole formed therethrough for receiving the peg. The peg may have one of a round shape, a square shape, and a polygonal shape.

In some aspects, the lateral mass fixation member includes a plate, the plate including a middle portion, an opening in the middle portion configured to allow passage of a screw to connect the plate with the facet implant member, and two tabs extending from opposite sides of the middle portion to contact lateral masses of adjacent vertebrae. The tabs may include one or more surface features, such as spikes, extending from the tabs for securing the tabs to the lateral masses of adjacent vertebrae. The tabs may include one or more holes formed therethrough to receive one or more fixation devices configured to secure the tabs to the lateral masses of adjacent vertebrae. The tabs may extend from the sides of the middle portion via hinges and the tabs are rotatable about the hinges relative to the middle portion. The lateral mass fixation member may include two tabs that extend from the facet implant member to contact lateral masses of adjacent vertebrae. The tabs may be moveable from a collapsed configuration for delivery of the system into a patient to an expanded configuration for attachment to lateral masses of adjacent vertebrae.

In some aspects, the system may further include a guide member for guiding at least one of the facet implant member or the lateral mass fixation member to a spine for attachment thereto.

In some aspects, the lateral mass fixation member includes a first face configured to attach to the facet implant member, and a second face coupled to and forming an angle with the first face, wherein the second face is configured to secure the lateral mass fixation member to lateral masses of adjacent vertebrae.

In some aspects, the lateral mass fixation member includes an anchor including an attachment device for attaching to the facet implant and a rod receiving member. The fixation member further includes a rod having a length sufficient to contact lateral masses of adjacent vertebrae.

A method for implanting a spinal fixation implant is disclosed. The method includes inserting a facet implant member in a facet joint and attaching a lateral mass fixation member to the facet implant member. In some embodiments, attaching the lateral mass fixation member includes screwing the lateral mass fixation member to the facet implant member. The method may further include attaching a guide rod to the facet implant member, positioning the lateral fixation member adjacent to the facet implant via the guide rod, and removing the guide rod. In some embodiments, inserting the facet implant includes delivering the facet implant via a guide tube.

A method for implanting a spinal fixation implant is disclosed. In one aspect, the method includes inserting a facet implant member of a fixation system in a facet joint, and expanding a lateral mass fixation member of the fixation system to contact lateral masses of adjacent vertebrae. In some aspects, the method further includes attaching the lateral mass fixation member to the lateral masses using at least one screw.

These and other aspects and embodiments will be described in further detail below, in reference to the attached drawing figures.

### BRIEF DESCRIPTION OF DRAWINGS

FIG. 1 is a lateral view of the C5 and C6 cervical vertebrae, illustrating the anatomy.

FIGS. 2A and 2B are posterior and lateral views, respectively, of a cervical spine with prior art posterior fixation devices applied thereto.

FIGS. 3A-3D are perspective (3A-3C) and lateral cross-sectional (3D) views of a portion of a cervical spine, illustrating a facet implant and a method for attaching a lateral mass fixation member thereto, according to one embodiment.

FIG. 4 is a perspective view of a portion of a cervical spine, illustrating a facet implant with a cylindrical attachment post and a lateral mass fixation member for attachment thereto, according to another embodiment.

FIGS. 5A and 5B are perspective and posterior views, respectively, of a portion of a cervical spine, illustrating a facet implant with a polygonal attachment post and a lateral mass fixation member for attachment thereto, according to another embodiment.

FIG. 6A is a perspective view of a jointed lateral mass fixation member, according to one embodiment.

FIGS. 6B and 6C are perspective views of a portion of a cervical spine, illustrating a method for attaching the jointed lateral mass fixation member of FIG. 6A to a facet implant, according to one embodiment.

FIG. 7A is a perspective view facet implant with pre-attached lateral mass fixation member, according to one embodiment.

FIGS. 7B-7D are lateral (7B) and perspective (7C-7D) views of a portion of a cervical spine, illustrating a method for implanting and attaching the facet implant with pre-attached lateral mass fixation members of FIG. 7A, according to one embodiment.

FIG. 8A is a perspective view facet implant with pre-attached lateral mass fixation members that are partially contained within the facet implant, according to one embodiment.

FIGS. 8B-8G are various views, some including a portion of a cervical spine, illustrating a method for implanting and attaching the facet implant with pre-attached lateral mass fixation members of FIG. 8A, according to one embodiment.

FIGS. 9A-9D are perspective views of a portion of a cervical spine, illustrating a method for advancing a folded lateral mass fixation member over a guide rod for attachment to a facet implant, according to one embodiment.

FIGS. 10A and 10B are perspective views of a lateral mass fixation member, according to one embodiment.

FIGS. 10C-10F are perspective views of a portion of a cervical spine, illustrating a method for attaching the lateral mass fixation member of FIGS. 10A and 10B to a facet implant, according to one embodiment.

FIGS. 11A-11H are various views of a cervical spine illustrating a system including a facet implant and a lateral mass fixation member and a method of attaching the lateral mass fixation member to the fact implant.

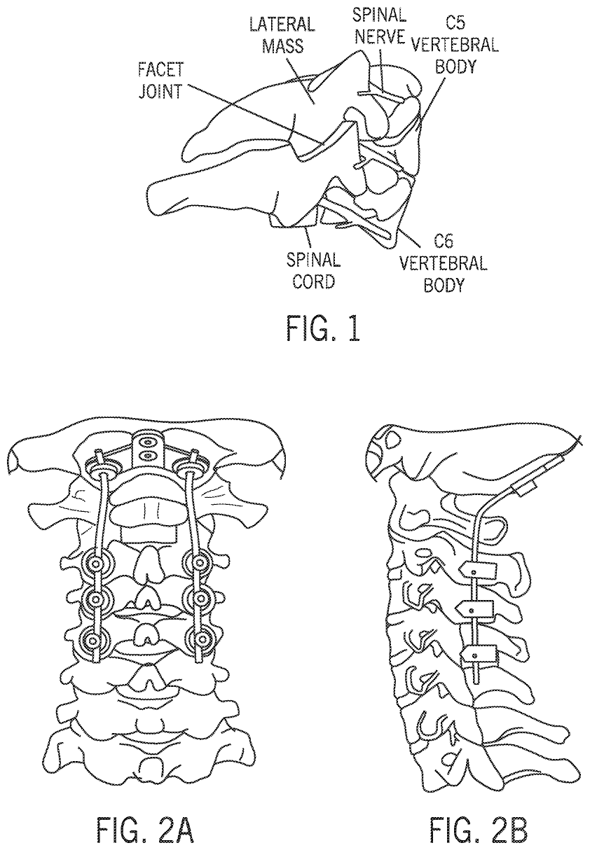

### DETAILED DESCRIPTION

The various embodiments described herein include a system for providing lateral mass fixation in the cervical spine, using posterior access, less invasive or minimally invasive, insertion methods. Generally, each system includes a facet component and a lateral mass fixation component. In some embodiments, the facet component and the lateral mass fixation component are separate devices until they are attached in situ within the patient. In other embodiments, the facet and lateral mass fixation components may be combined into one device. Some embodiments may include simply a facet implant by itself, which may be attached to one or more lateral mass fixation devices or members. Similarly, a lateral mass fixation device or member may be provided by itself, and may be compatible with one or more facet implant devices. The embodiments described herein also include methods for inserting, implanting and attaching the facet components and lateral fixation components described herein.

Lateral mass instrumentation may include a screw, staple or post in the lateral mass. Additional instrumentation such as a rod or plate may be used as a tension band to connect the rostral and caudal facet. This additional instrumentation may serve to limit flexion and extension as well as lateral bending. The facet implant connects with and/or helps guide the fixation member (or tension band) into place. The tension band device (e.g., plate, screw, rod or other material that bridges the rostral and caudal lateral mass) may be modular. It can be used to cross one or multiple motion segments. It may be put in before or after the facet implant. The combination of the facet implant and the lateral mass fixation member will provide superior fixation and stability of the joint in both the flexion and extension directions.

The surgeon may insert the facet implant though a minimal access incision, using an insertion system such as, but not limited to, the DTRAX® Spinal System, from Providence Medical Technology, Inc. (www.providencemt.com). A facet implant that may be used in the embodiments described herein includes the DTRAX® Cervical Cage, from Providence Medical Technology, Inc. (www.providencemt.com). The fixed point deep in the spine that is provided by the facet implant can be used to instrument the posterior cervical spine beyond the facet, from a percutaneous approach, without direct visualization. This avoids stripping all the soft tissue off the spine. A fixed point deep in the patient's spine prevents instruments from slipping off the spine and allows for location, alignment, and anchoring for fixation instrumentation. Also, the cervical facet has a fixed anatomic relationship to lateral mass bone consistent in all patients. Fixation instrumentation can be positioned off the facet implant to reliable landmarks on the lateral mass without direct visualization.

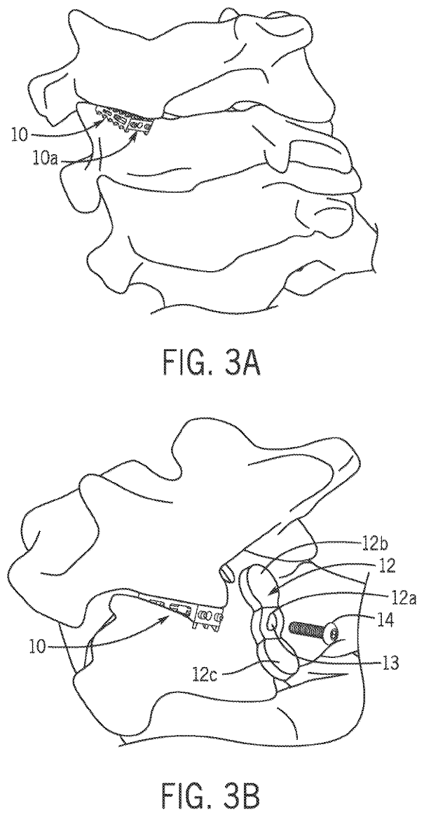

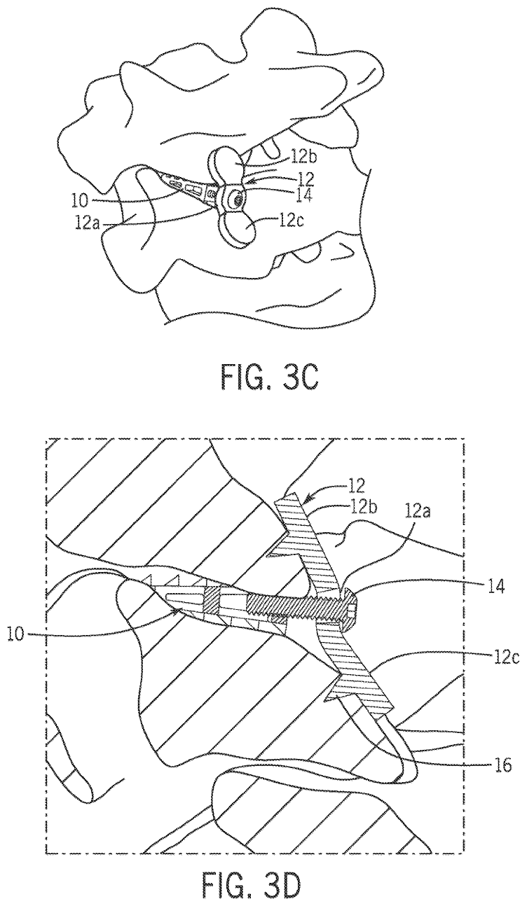

Referring now to FIGS. 3A-3D, in one embodiment, a lateral mass fixation system may include a facet implant 10, a lateral mass fixation member, such as a plate 12, and one or more screws 14 for attaching the plate 12 to the facet implant 10. The plate 12 (or “bar”) may extend over the rostral and caudal lateral mass and may be attached to the back of the facet implant 10 with the screw 14, which pulls the plate 12 up snugly against the lateral mass bone and provides additional fixation. As discussed in further detail below, the plate 12 may have a middle portion 12a and a two extensions 12b, 12c extending from the middle portion 12a. The middle portion 12a includes an aperture 13 configured to receive the screw 14 for attaching the plate 12 to the facet implant 10. The plate 12 may have a fixed shape or be adjustable by, for example a hinge between the middle portion 12a and the extensions 12b, 12c. In some embodiments, the extensions 12b, 12c may be flexible to allow the plate 12 to contour to the shape of the lateral mass bone. The facet implant 10 may include one or more threaded holes 10a formed through a distal end for receiving the one or more screws 14. In various embodiments, the screws may be tightened to a desired tension such that the plate 12 is securely attached to the facet implant 10 and the lateral mass bone. Referring to FIG. 3D, the plate 12 may include one or more surface features or spikes 16 (or alternatively screws or other attachment members) at the rostral and caudal end of the plate 12, which may penetrate into the lateral masses and help secure the plate 12 to the lateral masses. The facet implant 10, plate 12 and screw 14 may be made of any suitable biocompatible material, typically a metal or combination of metals.

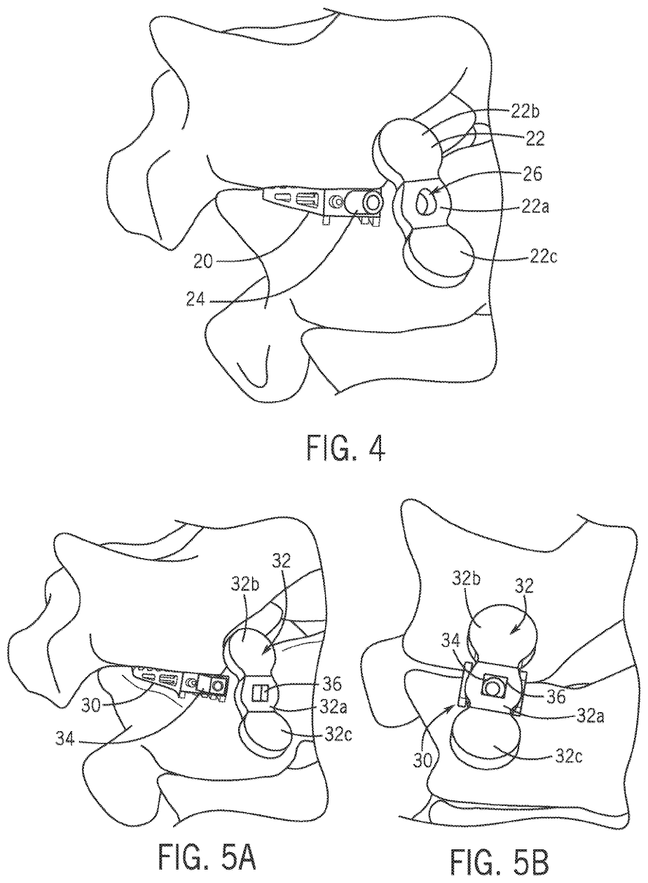

Referring to FIG. 4, in one embodiment, a lateral mass fixation system may include a facet implant 20 with a cylindrical peg 24 (or “post”) and a lateral mass fixation member, such as plate 22 with a corresponding circular opening 26 to fit around the peg 24. In various embodiments, the peg 24 may be located at a proximal end of the facet implant 20. The cylindrical peg 24 may have a hole formed therethrough which may be threaded to receive a screw (e.g., screw 14) to secure the plate 22 to the facet implant 20. As illustrated in this and other embodiments, the plate 22 may include a middle portion 22a and two extensions 22b, 22c extending at angles from the middle portion. The extension angles may be configured for fitting adjacent lateral masses. In some embodiments, the extensions 22b, 22c may be adjustable, relative to the middle portion 22a, by a physician.

In an alternative embodiment, and referring now to FIGS. 5A and 5B, a lateral mass fixation system may include a facet implant 30 with a polygonal peg 34 (or “post,” which is square in this embodiment but may have alternative shapes in other embodiments) and a lateral mass fixation member, such as plate 32 with a corresponding polygonal opening 36 to fit around or receive the peg 34. The polygonal peg 34 can orient the plate 32 to a pre-determined orientation relative to the axis of the facet implant 30, which can ensure proper contact between the plate and the lateral mass bones. As illustrated in this and other embodiments, the plate 32 may include a middle portion 32a and two extensions 32b, 32c extending at angles from the middle portion. The extension angles may be configured for fitting adjacent lateral masses. In some embodiments, the extensions 32b, 32c may be adjustable, relative to the middle portion 32a, by a physician.

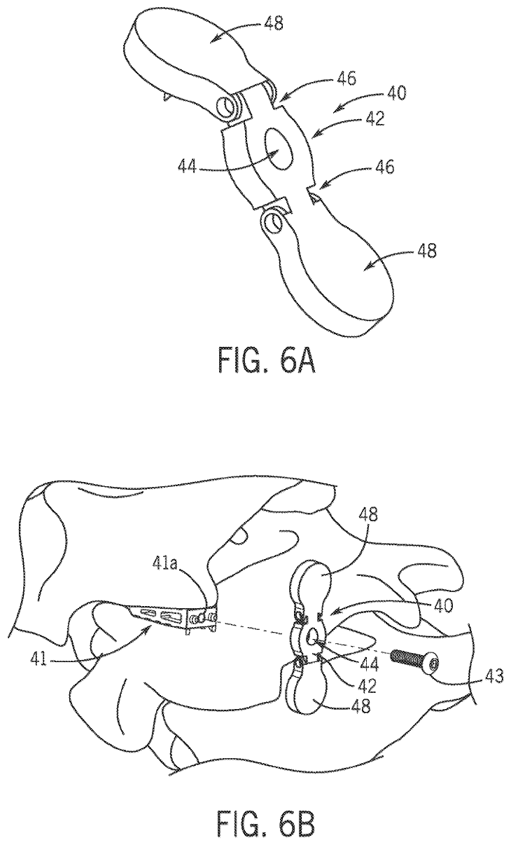

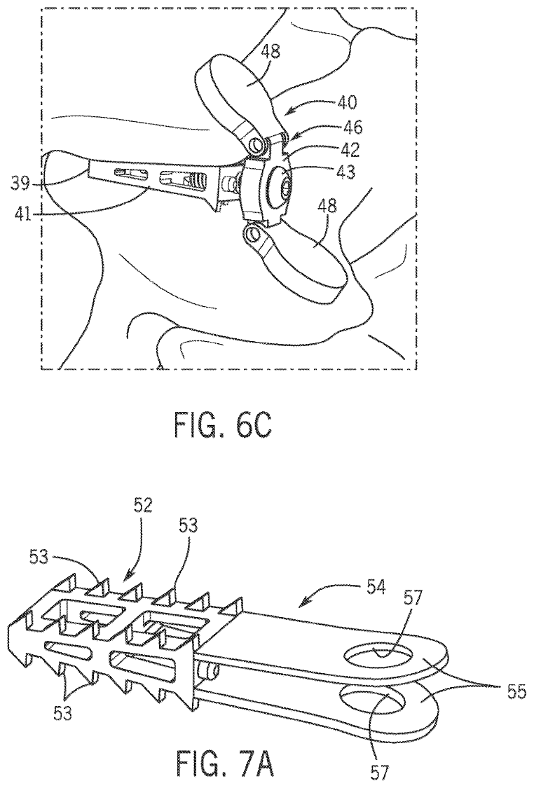

Referring now to FIGS. 6A-6C, in another embodiment, a lateral mass fixation system may include a lateral mass fixation member, such as a plate 40 that includes a middle portion 42 with a central opening 44, and two extensions 48 attached to opposite ends of the middle portion 42 via two hinges 46. The articulation of the extensions 48 about the hinges 46 will allow for adjustment of the plate 40 to fit various anatomical angles of the facet surface.

FIGS. 6B and 6C illustrate attachment of the plate 40 to a facet implant 41, using a screw 43. As can be understood from FIG. 6B, the facet implant 41 may be inserted into a facet joint 39 from a posterior access point. The plate 40 may also be inserted via a posterior access point concurrently with or subsequent to insertion of the facet implant 41. As shown in FIG. 6B, the plate 40 may have two extensions 48 which can rotate or articulate about the hinges 46. In various embodiments, the plate 40 may be inserted in a substantially linear configuration, as shown in FIG. 6B. A central opening or hole 44 through the plate 40 may be aligned with a complementary hole 41a in the facet implant 41 to receive the screw 43, which affixes the plate 40 to the facet implant 41. The extensions 48 may be rotated about the hinges 46 relative to the middle portion 42 to secure the plate 40 to the lateral masses. In various embodiments, the extensions 48 may be pre-rotated prior to insertion or may be adjusted after insertion.

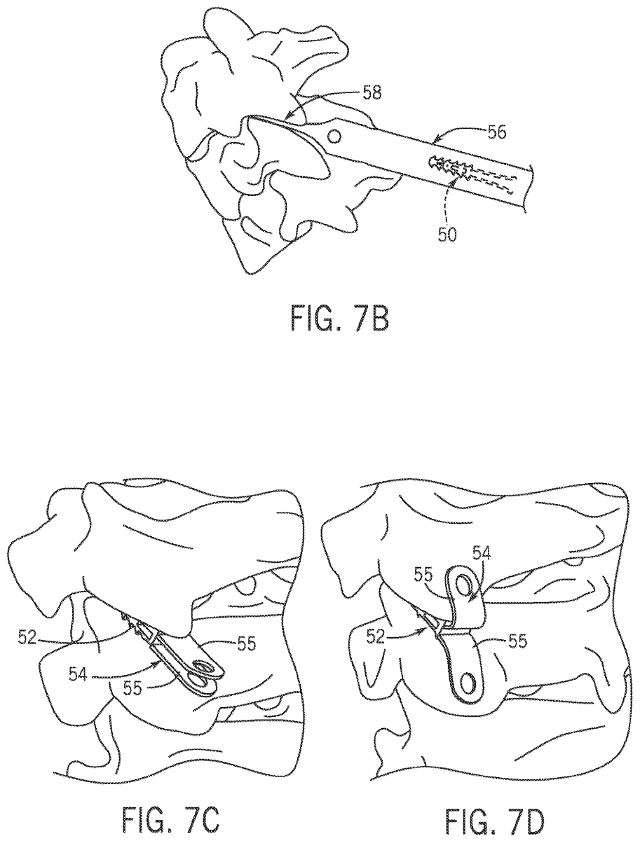

Referring now to FIGS. 7A-7D, in another embodiment, a lateral mass fixation system may include a combined facet implant/lateral mass fixation device 50. The device 50 may include a facet implant portion 52 and a lateral mass fixation member or portion 54, which are connected together before implantation in the patient. The facet implant portion 52 may include a number of teeth 53 for securing the facet implant portion 52 in the facet joint. In this embodiment, the lateral mass fixation portion 54 includes two flexible, semi-rigid, or rigid members (or “tabs”) 55 protruding or extending from a proximal end of the facet implant portion 52. The lateral mass fixation portion may include one or more holes 57 formed through the members 55 for receiving a securing device, such as a screw, for fixing the lateral mass fixation portions 52 to the lateral masses.

As illustrated in FIG. 7B, in one embodiment, the fixation device 50 may be advanced into the patient and into a facet joint 58 through an introducer device 56 (or “guide tube”). Referring to FIGS. 7C and 7D, once the facet implant portion 52 is implanted in the facet joint 58, the tabs 55 of lateral mass fixation portion 54 may be bent or otherwise moved to extend over the caudal and rostral lateral masses. In various alternative embodiments, the tabs 55 may move automatically by spring force, upon release from the introducer device 56, or may be bent manually or by mechanical actuation. The attachment of the lateral mass fixation portion 54 to the facet implant portion 52 allows the tabs 55 to be deployed in the desired location over the lateral masses.

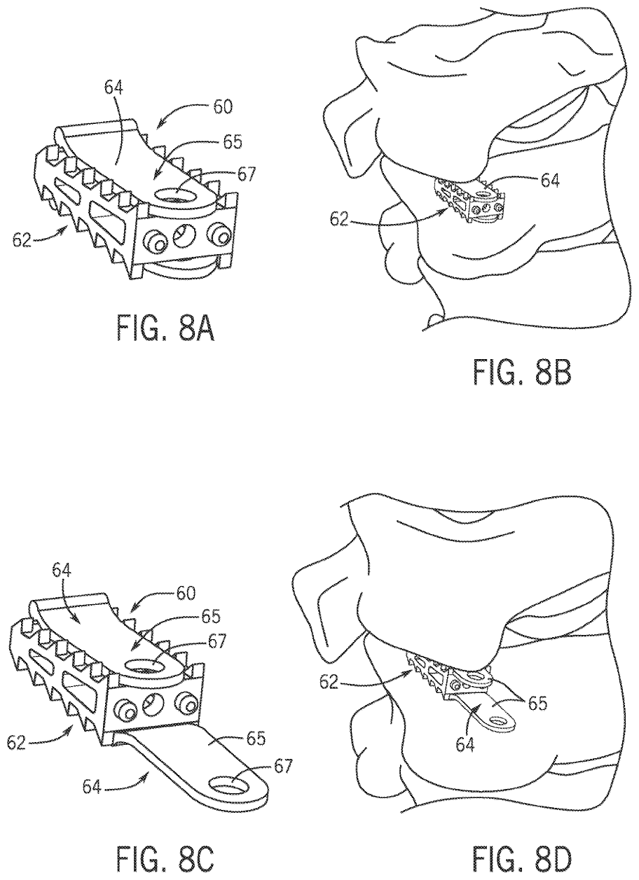

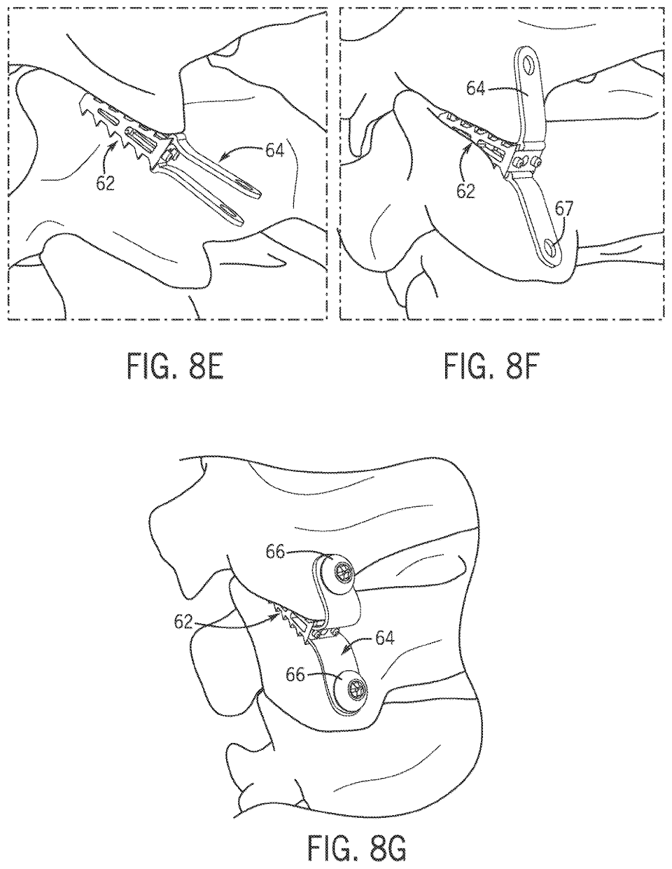

With reference now to FIGS. 8A-8G, in another alternative embodiment, a lateral mass fixation device 60 may include a facet implant portion 62 and a lateral mass fixation member or portion 64, which are connected together before implantation in the patient. In this embodiment, the lateral mass fixation portion 64 includes two flexible, semi-rigid, or rigid members (or “tabs”) 65 that are at least partially housed within the outer diameter of the facet implant portion 52 during delivery into the patient. The tabs 65 may include apertures or holes 67 defined therein for receiving a screw or other securing device. FIGS. 8A and 8B illustrate the fixation device 60 in its delivery configuration, with the tabs 65 housed within the facet implant portion 62. FIGS. 8C and 8D illustrate the fixation device 60 with one of the tabs 65 extending outward (proximally) from the facet implant portion 62. This extension motion may be achieved by sliding the tab 65 along a slot in the facet implant portion 62, for example.

FIG. 8E shows the fixation device 60 with both tabs 65 of the lateral mass fixation portion 64 extended. FIG. 8F shows the tabs 65 in a bent configuration to contact the caudal and rostral lateral masses. FIG. 8G shows the tabs 65 attached to the lateral masses via screws 66 received in the apertures 67.

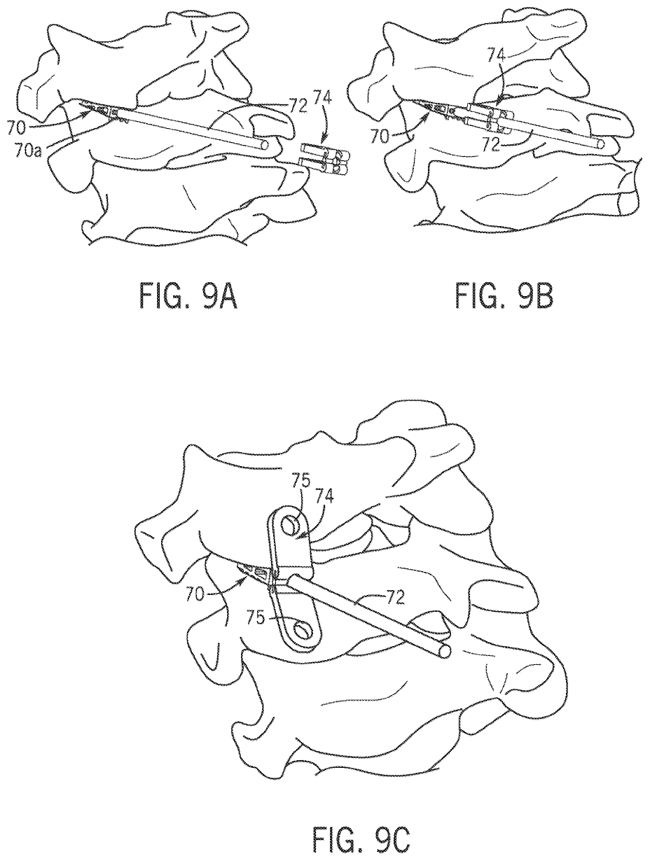

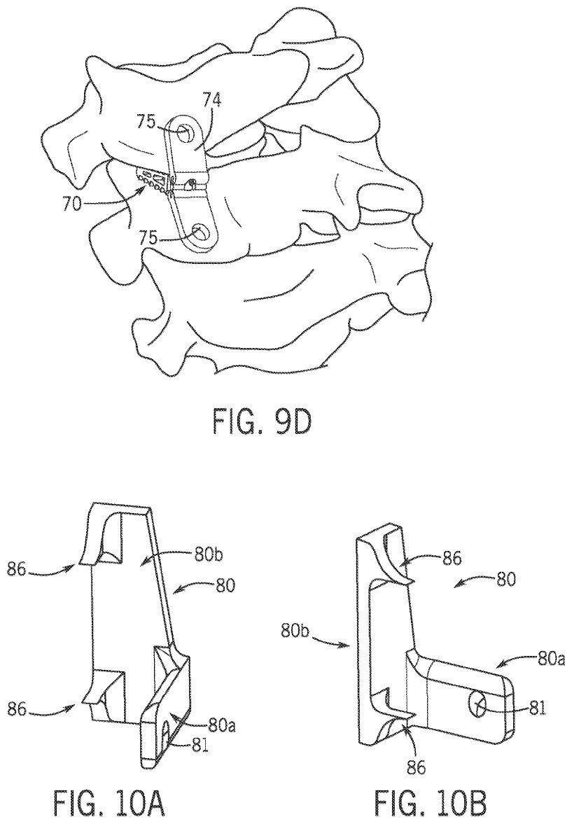

In yet another embodiment, and referring now to FIGS. 9A-9D, a lateral mass fixation system may include a facet implant 70, a guide rod 72 removably attached to the proximal end 70a of the facet implant 70, and a lateral mass fixation member 74 that is slidable over the guide rod 72. As illustrated in the figures, the lateral mass fixation member 74 may be advanced over the guide rod 72 in a collapsed configuration (FIGS. 9A and 9B) and then expanded to contact the lateral masses (FIG. 9C). The guide rod 72 may then be removed (FIG. 9D), leaving the facet implant 70 and lateral mass fixation member 74 in place. Screws (not shown) may be inserted through the holes 75 in the lateral mass fixation member 74 to attach the lateral mass fixation member 74 to the lateral masses.

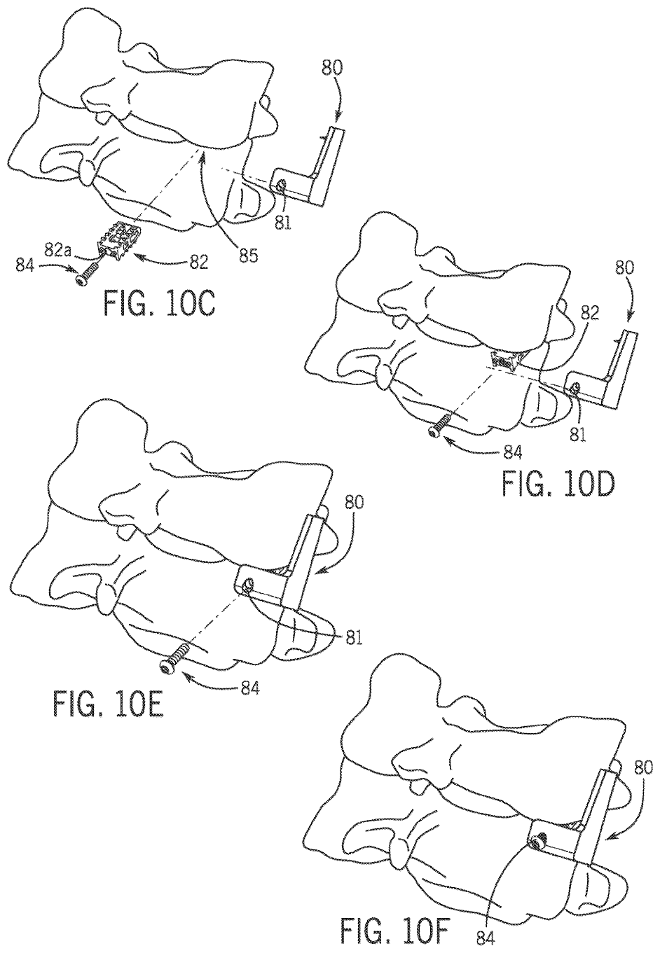

Referring now to FIGS. 10A-10F, in another embodiment, a lateral mass fixation member or plate 80 may include one or more surface features, such as spikes 86 for enhancing attachment to bone. The lateral mass fixation plate 80 may include two portions 80a, 80b, which may be arranged substantially perpendicular to one another. The first portion 80a may have a hole 81 formed therethrough to receive a screw or other attachment device. The second portion 80b may have the spikes 86 extending therefrom. The spikes 86 may extend substantially parallel to the first portion 80a. FIGS. 10C-10F illustrate a method for implanting a facet implant 82 and attaching the lateral mass fixation plate 80 to the facet implant 82 via a screw 84. The facet implant 82 may be inserted into the facet joint 85. The plate 80 may be inserted such that the hole 81 formed through the first portion 80a aligns with a hole 82a in the facet implant 82. The second portion 80b of the plate 80 may abut the lateral mass and secure the plate 80 and facet implant 82 in place when the screw 84 is inserted through the plate 80 into the facet implant 82. In an alternative embodiment, the lateral mass fixation plate 80 may have any of a number of different shapes, sizes, spikes or other surface features, and/or the like. Furthermore, in various embodiments, the lateral mass fixation plate 80 may be flexible, semi-rigid, or segmented.

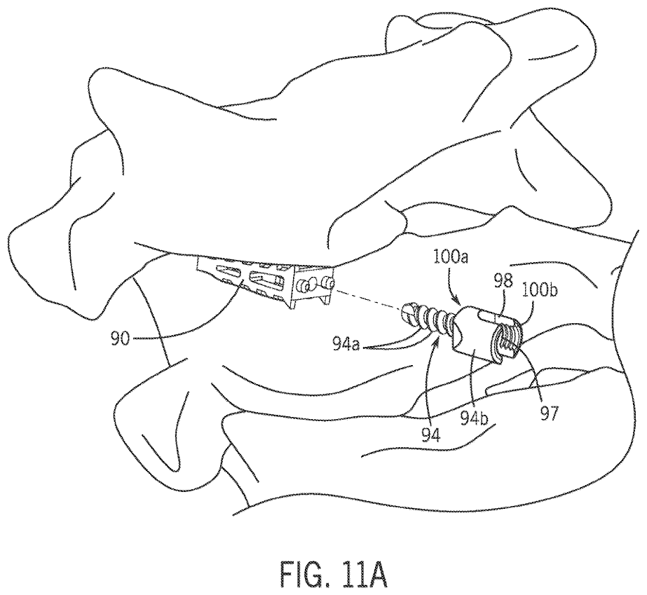

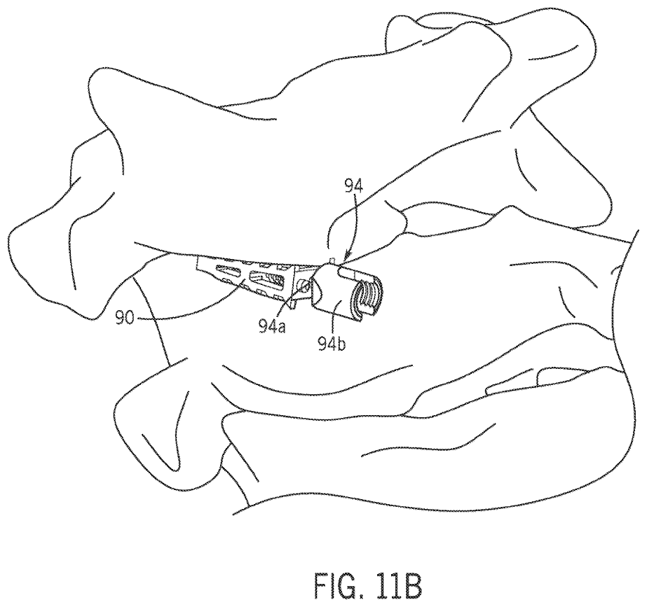

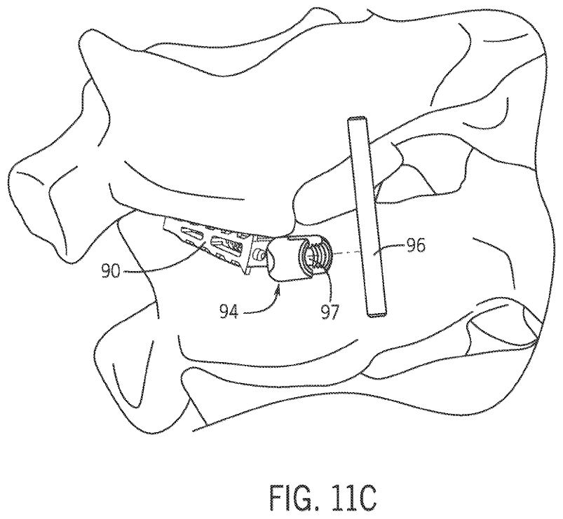

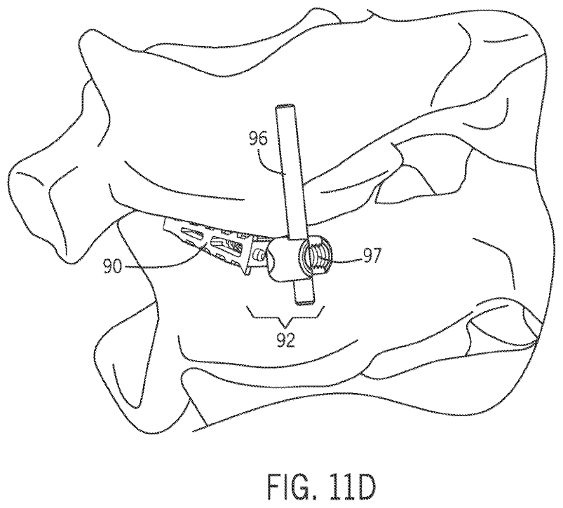

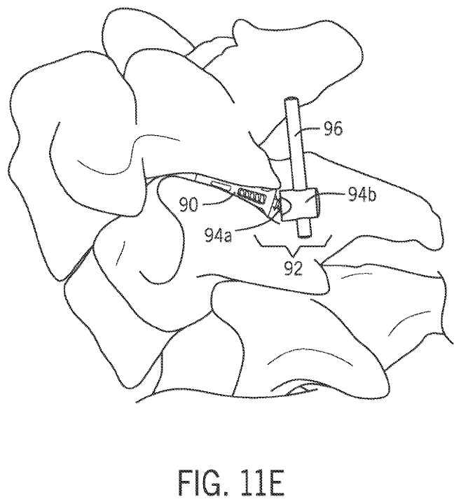

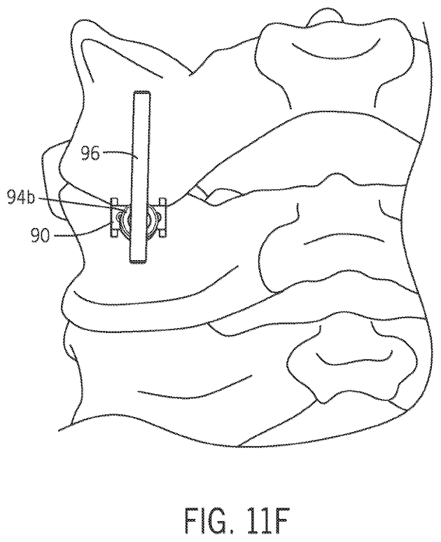

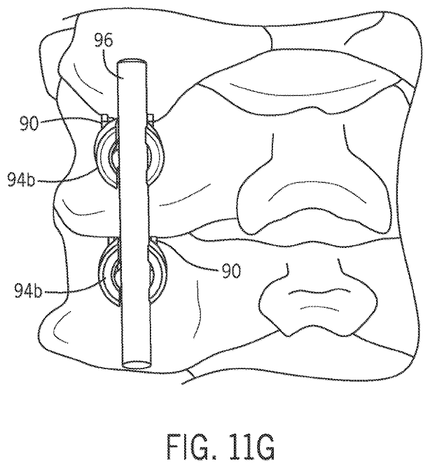

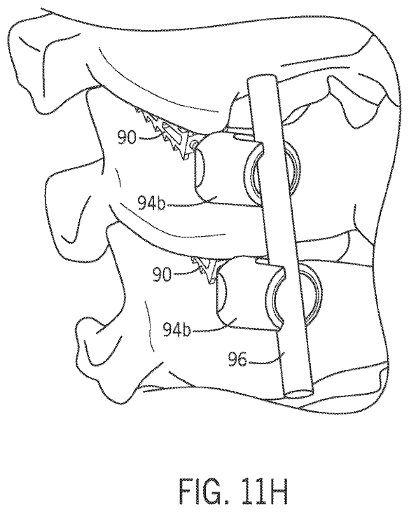

Referring now to FIGS. 11A-11C, in one embodiment, a lateral mass fixation system may include a facet implant 90 and a lateral mass fixation member 92 including an anchor 94 and a rod 96. As shown in FIG. 11A, and others, the anchor 94 includes an attachment device 94a, such as a screw, and a rod receiving member 94b. The rod receiving member 94b has a generally cylindrical body having proximal and distal ends 100a, 100b, with threads 97 and an open ended slot 98 sized to receive the rod 96 at the distal end 100b of the body. The rod 96 may extend across the facet joint and may be attached to the facet implant 90 via the anchor 94, which pulls the rod 96 up snugly against the lateral mass bone and provides additional fixation. FIG. 11B illustrates attachment of the anchor 94, via the attachment device 94a, to the implant 90, which has already been inserted in the facet joint. As can be understood from FIGS. 11C-11F, the rod 96 is introduced into the slot 98 of the rod receiving member 94b and secured therein. As indicated in FIGS. 11E-11F, which are side and posterior views of the facet implant 90 and lateral mass fixation member 92, the lateral mass fixation member 92 may be adjusted via rotation of the anchor 94 to secure or fix the rod 96 against the lateral masses. In some embodiments, as illustrated in FIGS. 11G-11H, which are posterior and posterior isometric views of the system, a plurality of facet implants 90 hold the rod 96 to stabilize the spine.

All relative and directional references (including: upper, lower, upward, downward, left, right, leftward, rightward, top, bottom, side, above, below, front, middle, back, vertical, horizontal, and so forth) are given by way of example to aid the reader's understanding of the particular embodiments described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other, unless specifically set forth in the claims.

Although the invention has been disclosed in the context of certain embodiments and examples, the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above.

What is claimed is:

1. A spinal implant system, comprising:
a facet implant member for positioning in a facet joint; and
a lateral mass fixation member attached to or attachable to the facet implant member.
